(19) United States
(12) Patent Application Publication
(10) Pub. No.: US 2015/0265139 A1
(43) Pub. Date: Sep. 24, 2015

(54) TUBE BODY FIXING STRUCTURE IN ENDOSCOPE CLEANING SHEATH, ENDOSCOPE CLEANING SHEATH WITH THE TUBE BODY FIXING STRUCTURE EMPLOYED THEREIN, AND ENDOSCOPE SYSTEM INCLUDING THE ENDOSCOPE CLEANING SHEATH

(71) Applicant: OLYMPUS CORPORATION, Tokyo (JP)

(72) Inventor: Keishiro SHINTANI, Tokyo (JP)

(73) Assignee: OLYMPUS CORPORATION, Tokyo (JP)

(21) Appl. No.: 14/734,327

(22) Filed: Jun. 9, 2015

Related U.S. Application Data
(63) Continuation of application No. PCT/JP2014/059761, filed on Apr. 2, 2014.

(30) Foreign Application Priority Data
May 16, 2013 (JP) 2013-104326

(51) Int. Cl. A61B 1/00; A61B 1/12

(52) U.S. Cl. CPC A61B 1/00135; A61B 1/00128; A61B 1/126

(57) ABSTRACT

A tube body fixing structure in an endoscope cleaning sheath includes: a flexible first tube body including a main lumen and a sub-lumen having a smaller diameter being provided inside along a longitudinal axis, the lumens each opening in the proximal end portion; a first fixing member including a fitting portion in which a proximal end portion of the main lumen is inserted, thereby the fitting portion being communicably connected to a proximal end portion of the main lumen; a second fixing member that is exteriorly mounted on the first tube body and includes a pressure contact portion that is biased against the fitting portion via the first tube body; and a flexible second tube body including a connection portion that is communicably connected to the sub-lumen on a proximal end side relative to the pressure contact portion, and an extension portion that extends from the connection portion.

### CROSS REFERENCE TO RELATED APPLICATION

This application is a continuation application of PCT/JP2014/059761 filed on Apr. 2, 2014 and claims benefit of Japanese Application No. 2013-104326 filed in Japan on May 16, 2013, the entire contents of which are incorporated herein by this reference.

### BACKGROUND OF THE INVENTION

1. Field of the Invention

This invention relates to a tube body fixing structure in an endoscope cleaning sheath, the tube body fixing structure reliably fixing a tube body such as a multi-lumen tube while keeping water tightness and gas tightness of the tube body, an endoscope cleaning sheath using the tube body such as a multi-lumen tube, and an endoscope system including the endoscope cleaning sheath.

2. Description of the Related Art

Conventionally, for endoscope systems each including an endoscope, various endoscope cleaning sheaths each including a configuration that sprays a mist mixture fluid toward front faces of, e.g., an observation window and an illumination window provided in a distal end face of an insertion portion of the endoscope to remove adherents or the like adhering to surfaces of the respective windows have been proposed and practically used.

A common configuration of a conventional endoscope cleaning sheath of this type mainly includes, e.g., a sheath insertion portion in which, e.g., an endoscope is to be inserted and set, a distal end nozzle portion provided on the distal end side of the sheath insertion portion, the distal end nozzle portion including a spray nozzle that ejects a mixture liquid obtained, by, for example, mixing and nebulization of a gas and a liquid, in order to clean away adherents or the like on an outer surface of, e.g., the observation window of, e.g., the set endoscope, and an operation portion provided on the proximal end side (hand side) of the sheath insertion portion, the operation portion being to be operated to supply the gas and the liquid to the spray nozzle.

Among the above, the sheath insertion portion is formed by a flexible tube having flexibility, and employs as the flexible tube, a multi-lumen tube including a main lumen having a large diameter in which, e.g., an endoscope is to be inserted, and a plurality of (at least two) sub-lumens for supplying the gas and the liquid, respectively, the sub-lumens each having a diameter that is smaller than that of the main lumen. Also, the operation portion is commonly formed by, for example, a rigid member such as a resin molding part or a metalwork part.

In such conventional endoscope cleaning sheath, in order to connect the operation portion and the sheath insertion portion (multi-lumen tube), for example, a form of connection in which a proximal end portion of the multi-lumen tube is held and thereby fixed between two parts included in the operation portion is commonly in practical use. The site of the connection between the operation portion and the sheath insertion portion (multi-lumen tube) generally requires ensuring a sufficient fixing strength and also ensuring water tightness and gas tightness between the parts. In addition, in order to make a fluid such as a gas or a liquid pass through each sub-lumen of the sheath insertion portion (multi-lumen tube), the endoscope cleaning sheath requires an ingenuity to prevent blockage of passage of the fluid such as a gas or a liquid in the sub-lumen in a state in which the proximal end portion of the multi-lumen tube is held between the two component parts of the operation portion, for example, an ingenuity in configuration such as providing, for example, a clearance space in a space around the sub-lumen so as not to crush the sub-lumen.

In this case, a configuration in which an elastic member is inserted between the multi-lumen tube and parts that press the multi-lumen tube to enhance the contact between the parts while the passage in each sub-lumen is ensured is considered.

For example, means disclosed in Japanese Patent Application Laid-Open Publication No. 2010-35621 is configured so that in a tube body fixing structure in which a retainer tube is connected and fixed to a proximal end side of a multi-lumen tube including a main lumen and sub-lumens, the retainer tube is inserted into the main lumen and a fastening tube is arranged on the outer side of the multi-lumen tube via a tapered tube. Here, the fastening tube is configured so as to be threadably joined and thereby fixed to the retainer tube.

In this configuration, with the multi-lumen tube kept between an outer peripheral face of the retainer tube and an inner peripheral face of the tapered tube, the fastening tube biases the tapered tube. Consequently, the multi-lumen tube is biased toward and fixed to the outer peripheral face of the retainer tube by the tapered tube. Here, a fluid supply pipe having elasticity (elastic body) is inserted into and fixed to the proximal end side of the sub-lumen, and thus, even if the multi-lumen tube is held between the outer peripheral face of the retainer tube and the inner peripheral face of the tapered tube, each sub-lumen is prevented from being crushed, ensuring a flow of a fluid.

### SUMMARY OF THE INVENTION

A tube body fixing structure in an endoscope cleaning sheath according to an aspect of the present invention includes: a flexible first tube body including a proximal end portion and a distal end portion, a main lumen and a sub-lumen having a diameter that is smaller than that of the main lumen being provided inside along a longitudinal axis, the lumens each opening in the proximal end portion; a first fixing member including a fitting portion inserted in and thereby communicably connected to a proximal end portion of the main lumen; a second fixing member that is exteriorly mounted on the first tube body and includes a pressure contact portion that is biased against the fitting portion via the first tube body; and a flexible second tube body including a connection portion that is communicably connected to the sub-lumen on a proximal end side relative to the pressure contact portion, and an extension portion that extends from the connection portion.

An endoscope cleaning sheath according to an aspect of the present invention includes the tube body fixing structure in an endoscope cleaning sheath, and an insertion portion of an endoscope can be inserted in the main lumen via a communication hole, and a distal end nozzle portion that includes a spray nozzle that can feed a liquid to a front-side outer surface of the endoscope and is communicably connected to the sub-lumen is provided at the distal end portion of the tube body.

An endoscope system according to an aspect of the present invention is an endoscope system including an endoscope and a light source apparatus, the endoscope system including: the endoscope cleaning sheath; and a tubular member through which the endoscope cleaning sheath is to be inserted.

### BRIEF DESCRIPTION OF THE DRAWINGS

FIG. 1 is a schematic configuration diagram of an endoscope cleaning sheath with a tube body fixing structure according to an embodiment of the present invention employed therein;

FIG. 2 is an enlarged cross-sectional view of a sheath insertion portion (multi-lumen tube) of the endoscope cleaning sheath in FIG. 1;

FIG. 3 is a main part enlarged perspective diagram illustrating a site of connection between the sheath insertion portion (multi-lumen tube) of the endoscope cleaning sheath in FIG. 1 and an operation portion;

FIG. 4 is a front view of the endoscope cleaning sheath in FIG. 1;

FIG. 5 is a cross-sectional view along line [5]-[5] in FIG. 4;

FIG. 6 is a cross-sectional view along line [6]-[6] in FIG. 4; and

FIG. 7 is a main part enlarged view of the site indicated by [7] in FIG. 6.

### DETAILED DESCRIPTION OF THE PREFERRED EMBODIMENT

The present invention will be described below by means of the illustrated embodiment.

An embodiment, which will be described below, is one exemplifying, as a medical tool with a tube body fixing structure according to the present invention employed therein, for example, an endoscope cleaning sheath including a sheath insertion portion including a tube body in which an insertion portion of an endoscope can be inserted, an operation portion connected to a proximal end of the sheath insertion portion, and a distal end nozzle portion connected to the distal end of the sheath insertion portion, the distal end nozzle portion including a spray nozzle for cleaning away adherents or the like on a front-side outer surface of each of an observation window and an illumination window of, e.g., an endoscope inserted in the tube body (sheath insertion portion), and an endoscope system using the endoscope cleaning sheath.

Note that in each of the drawings used for the below description, components may be illustrated on difference scales so that the respective components have sizes that are large enough to be recognized in the drawing, and the present invention is not limited only to the counts and amounts, and the shapes of the components, and the size ratios and the relative positional relationships among the components illustrated in the drawings.

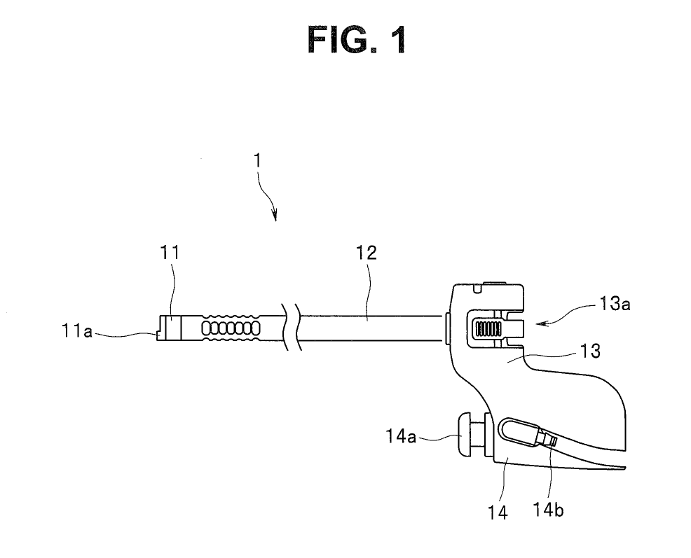

A configuration of an endoscope cleaning sheath with a tube body fixing structure according to an embodiment of the present invention employed therein will be described below with reference to FIGS. 1 to 7.

FIG. 1 illustrates an overall schematic configuration of an endoscope cleaning sheath 1 with the tube body fixing structure according to the present embodiment employed therein. The endoscope cleaning sheath mainly includes, e.g., a distal end nozzle portion 11, a sheath insertion portion 12, which is a flexible tube body and is also a first tube body, an operation portion 13, and a control switch portion 14.

The distal end nozzle portion 11 receives a distal end part of the sheath insertion portion 12 and is thereby connected to the distal end part of the sheath insertion portion 12 in an integrated manner The distal end nozzle portion 11 is formed so as to have a substantially-cylindrical shape in its entirety, by a rigid member such as a rigid resin or a metal member.

On the front side of the distal end nozzle portion 11, a spray nozzle 11a for ejecting a nebulized mixture liquid for cleaning away adherents adhering to front-side outer surfaces of, e.g., an observation window and an illumination window (not illustrated) in a distal end face of an endoscope (not illustrated) inserted and set in the sheath insertion portion 12 is disposed in an integrated manner.

The distal end nozzle portion 11 is connected to the distal end side of the sheath insertion portion 12 in an integrated manner, and the operation portion 13 is connected to the proximal end side of the sheath insertion portion 12 in an integrated manner. As the sheath insertion portion 12, what is called a multi-lumen tube including a large-diameter conduit to which an endoscope insertion portion (not illustrated) is to be inserted and a plurality of (two in the present embodiment) conduits through which a gas and a fluid for ejecting a nebulized mixture liquid from the spray nozzle 11a of the distal end nozzle portion 11 pass, the multi-lumen tube being formed in a tube shape in its entirety using a flexible material, is employed.

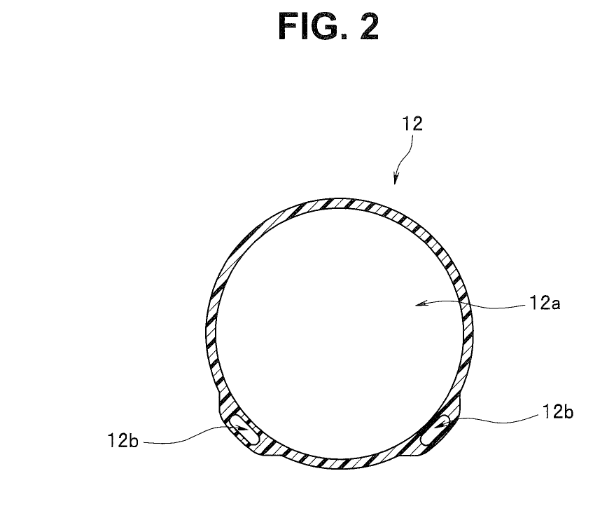

The sheath insertion portion 12 has a cross-sectional shape, for example, such as illustrated in FIG. 2. In other words, the sheath insertion portion 12 includes one main lumen 12a, which is the large-diameter conduit to which an endoscope insertion portion (not illustrated) is inserted, and a plurality of (two in the present embodiment) sub-lumens 12b formed in sites close to an outer periphery of the main lumen 12a. One of the two sub-lumens 12b is a gas feeding conduit that allows a gas to pass therethrough, and the other is a liquid feeding conduit that allows a liquid such as water to pass therethrough. As illustrated in FIG. 2, the parts in which the two sub-lumens 12b are formed are formed so as to slightly project outward in a radial direction. Consequently, the sheath insertion portion 12 has an outer peripheral shape including an outer peripheral portion having a roughly round shape in its entirety in cross-section and a plurality of (two) protrusion portions on parts of the outer peripheral portion.

As described above, the sheath insertion portion 12 is formed using a flexible material, and thus, if an external force is applied from an outer surface thereof to the sites in which the sub-lumens 12b are formed (the protrusion portions), for example, the respective conduits formed by the sub-lumens 12b are deformed in such a manner that the respective conduits are crushed; however, the cross-sectional shape of the sheath insertion portion 12 is maintained to be close to the roughly round shape in its entirety. Here, in order to prevent a problem from occurring even if the respective conduits are crushed, the sheath insertion portion 12 has flexibility.

For brief description, the sheath insertion portion 12, which is formed by a multi-lumen tube, includes a proximal end portion and a distal end portion, is formed so as to have flexibility, and includes a main lumen 12a, and sub-lumens 12b each having an inner diameter that is smaller than that of the main lumen 12a, provided inside along a longitudinal axis, and the lumens 12a and 12b each open in a proximal end portion.

Inside the distal end nozzle portion 11, flow passages (not illustrated) to which, when the distal end nozzle portion 11 and the sheath insertion portion 12 are connected, the respective conduits of the two sub-lumens 12b in the sheath insertion portion 12 are connected respectively, are formed. Furthermore, the respective flow passages are merged into a single flow pass inside the distal end nozzle portion 11, which ultimately communicates with the spray nozzle. With this configuration, a gas and a liquid flowing via the two sub-lumens 12b in the sheath insertion portion 12 are mixed and nebulized inside the distal end nozzle portion 11 and then sprayed from an ejection port (not illustrated) of the spray nozzle 11a toward, e.g., an observation window and an illumination window in a distal end face of an endoscope (not illustrated) inserted through the endoscope cleaning sheath 1.

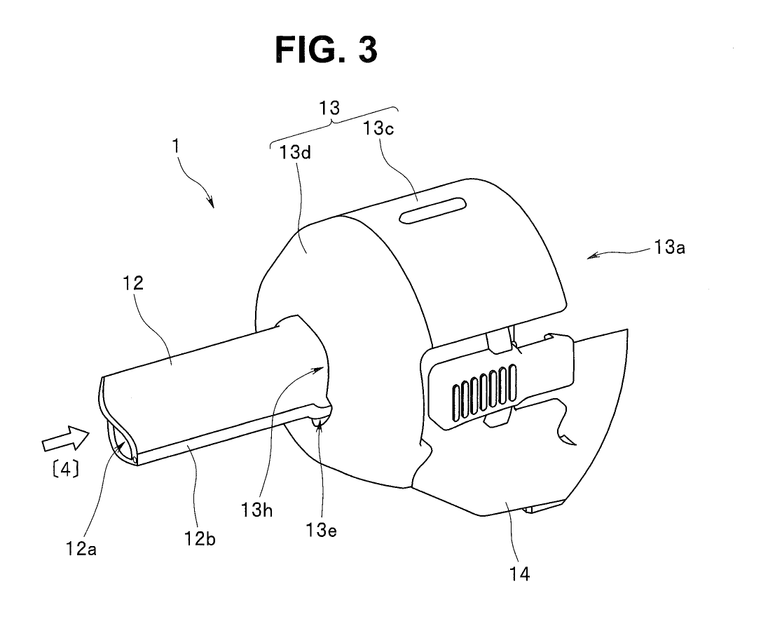

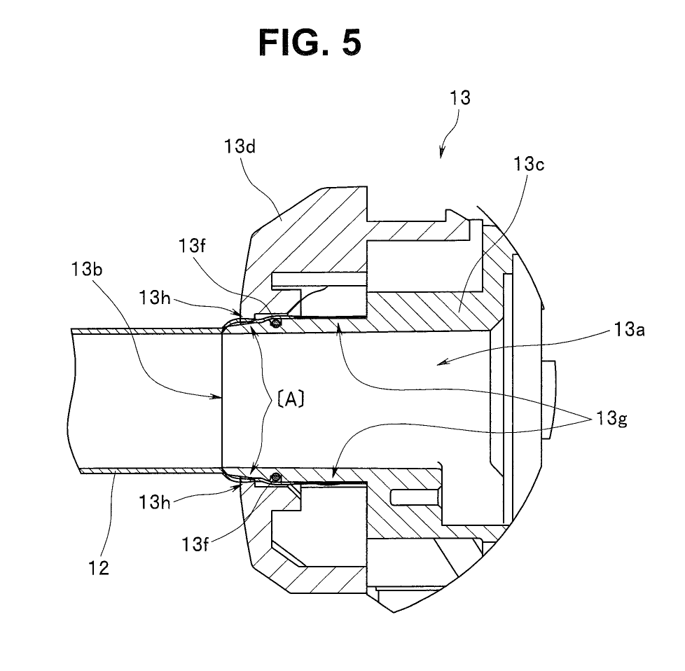

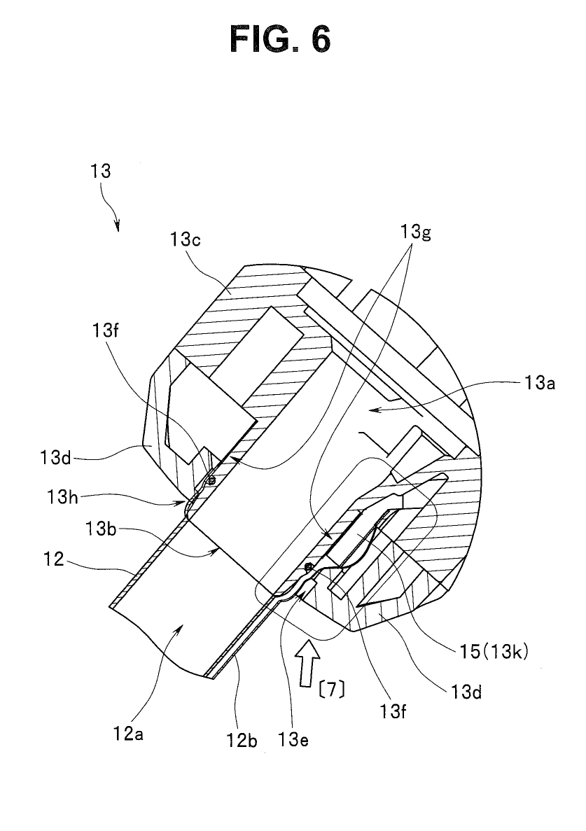

The operation portion 13 is provided so as to be continuous with the proximal end side of the sheath insertion portion 12. The operation portion 13 is formed by two component members, that is, a first body portion 13c, which is a first fixing member, and a second body portion 13d, which is a second fixing member. In the first body portion 13c forming the proximal end side of the operation portion 13, a proximal end opening 13a (see FIG. 3) for allowing the endoscope insertion portion (not illustrated) to be inserted in the main lumen 12a of the sheath insertion portion 12 is formed. The proximal end opening 13a is a proximal end opening of a barrel shape portion 13g (see FIGS. 5 and 6) (communication hole) for forming a space extending through the first body portion 13c. Furthermore, a distal end opening 13b is formed on the distal end side of the barrel shape portion 13g. In other words, the proximal end opening 13a and the distal end opening 13b communicate with each other via a predetermined space in the operation portion 13. A proximal end portion of the main lumen 12a in the sheath insertion portion 12 is press-fitted onto and fixed to the outer peripheral side of the barrel shape portion 13g so as to be in close contact with the outer peripheral side of the barrel shape portion 13g. In other words, the barrel shape portion 13g is formed as a fitting portion for communicably connecting the proximal end portion of the main lumen 12a to the operation portion 13.

On the other hand, in the second body portion 13d forming the distal end side of the operation portion 13, an opening 13h having a diameter dimension that enables the barrel shape portion 13g with the proximal end portion of the sheath insertion portion 12 pressed and fixed thereon to be inserted to the opening 13h is formed.

Then, when the first body portion 13c with the proximal end portion of the sheath insertion portion 12 pressed and fixed on the barrel shape portion 13g, and the second body portion 13d are combined, the proximal end portion of the sheath insertion portion 12 is fixed in such a manner that the proximal end portion of the sheath insertion portion 12 is held between the first body portion 13c and the second body portion 13d (see FIG. 5). In other words, the second body portion 13d is arranged so as to be exteriorly mounted on the sheath insertion portion 12. In this state, the second body portion 13d is arranged so as to be biased against the barrel shape portion 13g via the sheath insertion portion 12. In this case, a site in which the second body portion 13d is biased against the barrel shape portion 13g via the sheath insertion portion 12 is referred to as a pressure contact portion (see symbol [A] in FIG. 5). Note that the pressure contact portion A is a site corresponding to an inner peripheral face of the opening 13h in the second body portion 13d.

Inside the operation portion 13, an O-shaped ring 13f, which is an elastic member, is disposed on an outer peripheral face of the barrel shape portion 13g of the first body portion 13c. Therefore, water tightness and gas tightness of the first body portion 13c and the second body portion 13d are ensured with the main lumen 12a held therebetween. In other words, the O-shaped ring 13f, which is a water tightness keeping section water-tightly connected between the barrel shape portion 13g (fitting portion) and the main lumen 12a, is disposed between the barrel shape portion 13g (fitting portion) and the main lumen 12a.

The water tightness keeping section is provided between the pressure contact portion A (the site in which the second body portion 13d is in pressure contact with the barrel shape portion 13g via the sheath insertion portion 12) and portions of connection between the sub-lumens 12b and connection tubes 15 to be described later in a long axis direction of the sheath insertion portion 12.

This configuration enables, when an endoscope insertion portion (not illustrated) is inserted from the proximal end opening 13a, the endoscope insertion portion to be inserted through the predetermined space in the barrel shape portion 13g of the operation portion 13 and then inserted to the main lumen 12a of the sheath insertion portion 12 from the distal end opening 13b.

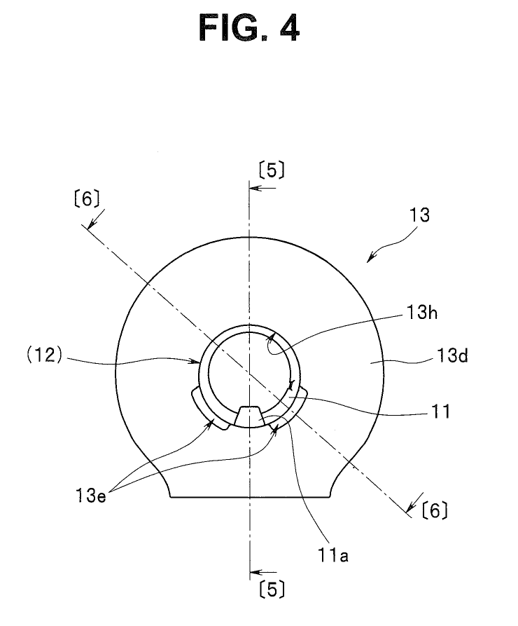

Here, as illustrated in FIG. 4, a shape of the opening 13h of the second body portion 13d as viewed from the front side is a roughly round shape in its entirety so as to conform to the main lumen 12a, and a plurality of cutout portions 13e are formed in predetermined areas substantially corresponding to the positions and shapes of the plurality of (two) sub-lumens 12b, the cutout portions 13e projecting outwardly in a radial direction of the opening 13h, at a plurality of sites (two sites in the present embodiment) on an outer peripheral face thereof In other words, the cutout portions 13e are formed in the pressure contact portion A of the second body portion 13d so as to each have a shape that can prevent the sub-lumens 12b from being biased.

Therefore, as illustrated in FIG. 5, an outer peripheral face of the proximal end portion of the sheath insertion portion 12 is held and swaged between the inner peripheral face of the opening 13h of the second body portion 13d and the outer peripheral face of the barrel shape portion 13g of the first body portion 13c, ensuring water tightness and gas tightness, and is fixed in close contact in the pressure contact portion A (see FIG. 5).

On the other hand, as illustrated in FIG. 6, the respective sub-lumens 12b in the proximal end portion of the sheath insertion portion 12 are disposed so as to be received in the (two) respective corresponding cutout portions 13e. Consequently, each sub-lumen 12b is fixed between the second body portion 13d and the first body portion 13c inside the operation portion 13 without being crushed by the members 13d and 13c and with the flow passage secured.

Furthermore, inside the operation portion 13, a plurality of (two) connection tubes 15, which are second tube bodies having flexibility and a short barrel shape are fixedly arranged at respective predetermined positions on the outer side of the barrel shape portion 13g of the first body portion 13c. The two connection tubes 15 are provided so as to correspond to the two sub-lumens 12b, respectively. These connection tubes 15 are pressed into and fixed to proximalmost end portions of the respective sub-lumens 12b at connection portions on the proximal end side relative to the pressure contact portion A (see FIG. 5), and are thereby communicably connected to the proximalmost end portions of the respective sub-lumens 12b. Each connection tube 15 includes an extension portion extending from the relevant connection portion, and a proximal end of the extension portion is connected to a later-described control switch portion 14.

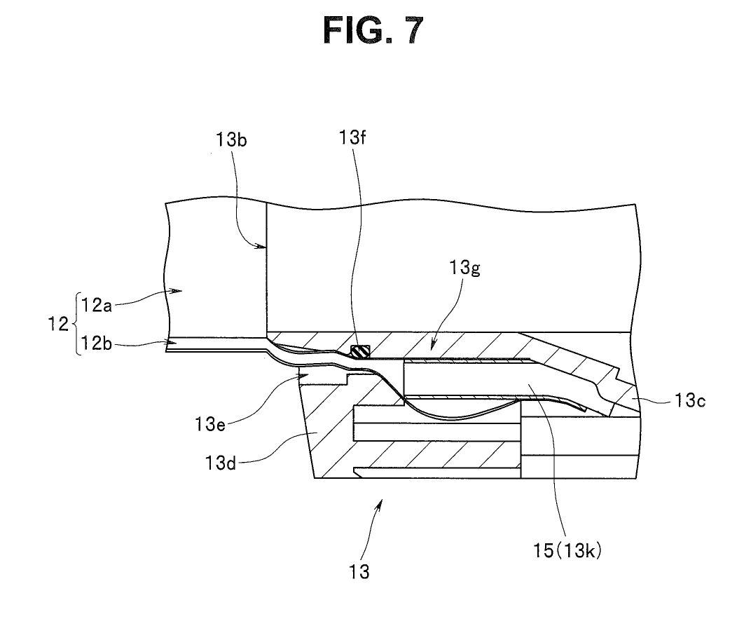

Note that the extension portions of the connection tubes 15 are fixedly arranged in respective communication grooves 13k (see FIGS. 6 and 7), which are predetermined spaces on the outer side of the first body portion 13c (first fixing member) or the inner side of the second body portion 13d (second fixing member).

The control switch portion 14 is formed integrally with the operation portion 13, and includes, e.g., an operation switch 14a that controls gas feeding operation and spraying operation. In the control switch portion 14, a plurality of connection fittings 14b (only one of the connection fittings is illustrated in FIG. 1) to which distal ends of an air feeding tube and a liquid feeding tube (not illustrated) extending from a gas/liquid feeding apparatus are to be connected. Inside the control switch portion 14, flow passages from the connection fittings 14b to the two sub-lumen 12b of the sheath insertion portion 12 connected to the operation portion 13, via the operation switch 14a, are formed (not illustrated).

Note that the operation switch 14a is formed by, for example, a two-stage switch. For example, a gas feeding operation is performed by a first-stage pressing operation of the operation switch 14a. When an operation to further press the operation switch 14a down (second-stage pressing operation) from this state (first-stage pressing operation state) is performed, a liquid feeding operation is performed in addition to the gas feeding operation. Consequently, a nebulized mixture liquid is sprayed from the ejection port of the spray nozzle 11 a of the distal end nozzle portion 11.

The tube body fixing structure according to the present embodiment is configured as briefly described below. In other words, the proximal end portion of the multi-lumen tube (sheath insertion portion 12), which is a tube body, is fixed in such a manner that the proximal end portion of the multi-lumen tube is held between two parts included in the operation portion 13, that is, the first body portion 13c and the second body portion 13d. Here, water tightness and gas tightness of the part between the first body portion 13c and the second body portion 13d are ensured by the O-shaped ring 13f, which is an elastic member. Also, the plurality of sub-lumens 12b in the multi-lumen tube (sheath insertion portion 12) are arranged in the respective cutout portions 13e formed in the second body portion 13d, whereby the flow passages of the respective sub-lumens 12b are secured. Furthermore, inside the operation portion 13, the connection tubes 15 are fixedly pressed in the proximalmost end parts of the respective sub-lumens 12b. The connection tubes 15 are fixedly arranged between the first body portion 13c and the second body portion 13d. Here, the connection tubes 15 are arranged so as not to overlap a position at which the O-shaped ring 13f is disposed. The configuration secures the flow passages between the respective sub-lumens 12b and the operation switch 14a.

The endoscope cleaning sheath 1 with the above-described tube body fixing structure according to the present embodiment employed therein is used in combination with an endoscope system including an endoscope. For the endoscope system including an endoscope, an ordinary type of endoscope system that is practically used conventionally is employed. An example configuration of the endoscope system is a system formed by, e.g., an endoscope and a light source apparatus, the system including, e.g., a trocar including a tubular member through which the endoscope cleaning sheath 1 with an endoscope inserted therein is to be inserted.

Here, operation when the endoscope cleaning sheath 1 is used will briefly be described below. First, an endoscope insertion portion of an endoscope is inserted from the proximal end opening 13 a of the operation portion 13 in the endoscope cleaning sheath 1 toward the inside of the operation portion 13. Then, the endoscope insertion portion is inserted through the inside of the barrel shape portion 13g in the operation portion 13 and projects from the distal end opening 13b. The main lumen 12a of the sheath insertion portion 12 is connected to the distal end opening 13b. Therefore, the endoscope insertion portion is inserted through the inside of the main lumen 12a of the sheath insertion portion 12 and ultimately reaches a site near the distal end nozzle portion 11. In such a manner as described above, the endoscope insertion portion enters a state in which the endoscope insertion portion is inserted in the endoscope cleaning sheath 1. The endoscope cleaning sheath 1 in this state is inserted and set in, for example, the trocar. This state enables, e.g., observation of the inside of a body cavity using the endoscope. Then, in such case, if an endoscopic observation image is disturbed by adherents or the like adhering to a front-side outer surface thereof, the operation switch 14a of the control switch portion 14 is operated to spray a gas or a nebulized mixture liquid from the ejection port of the spray nozzle 11a in the distal end nozzle portion 11. Consequently, the adherents or the like on the front-side outer surface of the endoscope are removed.

As described above, according to the above-described embodiment, a fixing position at which the main lumen 12a of the sheath insertion portion 12 (multi-lumen tube) is fixed so as to be held between the two parts, which are the first body portion 13c and the second body portion 13d of the operation portion 13, and a position at which the O-shaped ring 13f, which is an elastic member, are set to be positions that are different from each other in the axis direction of the sheath insertion portion 12. Also, the O-shaped ring 13f (elastic member) is arranged on the side that applies no external force to the sheath insertion portion 12 (multi-lumen tube), that is, the first body portion 13c side. This configuration enables water tightness and gas tightness of the main lumen 12a to be kept without the main lumen 12a being affected by expansion/contraction of the sheath insertion portion 12.

Also, the sites of press-fit connection between the proximal ends of the sub-lumens 12b in the sheath insertion portion 12 and the connection tubes 15 are arranged in sites that does not cover (are not positioned over) the O-shaped ring 13f. This configuration enables ensuring water tightness and gas tightness of the inside of the operation portion 13 while securing the flow passages of the sub-lumens 12b.

Therefore, in the structure of fixing between the sheath insertion portion 12, which is a tube body and employs a multi-lumen tube, and the operation portion 13 in the endoscope cleaning sheath 1 according to the present embodiment, the sheath insertion portion 12 is reliably fixed to the operation portion 13 while the flow passages of the plurality of sub-lumens 12b in the sheath insertion portion 12 are secured, enabling maintenance of water tightness and gas tightness of the inside of the operation portion 13.

Note that the present invention is not limited to the above-described embodiment and various alterations and applications are possible without departing from the spirit of the invention. Furthermore, the above-described embodiment includes various phases of the invention, and various aspects of the invention may be extracted by arbitrary combinations of the plurality of elements disclosed. For example, even where some elements are deleted from all the elements indicated in the above embodiment, a configuration with such elements deleted may be extracted as an aspect of the invention if such configuration can solve a problem to be solved by the invention and provide an effect of the invention.

What is claimed is:

1. A tube body fixing structure in an endoscope cleaning sheath, comprising:
a flexible first tube body including a proximal end portion and a distal end portion, a main lumen and a sub-lumen having a diameter that is smaller than that of the main lumen being provided inside along a longitudinal direction, the lumens each opening in the proximal end portion;
a first fixing member including a fitting portion inserted in and thereby communicably connected to a proximal end portion of the main lumen;
a second fixing member that is exteriorly mounted on the first tube body and includes a pressure contact portion that is biased against the fitting portion via the first tube body; and
a flexible second tube body including a connection portion that is communicably connected to the sub-lumen on a proximal end side relative to the pressure contact portion, and an extension portion that extends from the connection portion.

2. The tube body fixing structure in an endoscope cleaning sheath according to claim 1, comprising a water tightness keeping section that water-tightly interconnects the fitting portion and the main lumen.

3. The tube body fixing structure in an endoscope cleaning sheath according to claim 2, wherein the water tightness keeping section is provided between the pressure contact portion and the connection portion in a longitudinal axis direction.

4. The tube body fixing structure in an endoscope cleaning sheath according to claim 2, wherein the water tightness keeping section is an O-shaped ring.

5. The tube body fixing structure in an endoscope cleaning sheath according to claim 1, wherein the pressure contact portion includes a cutout portion that can prevent the sub-lumen from being biased.

6. The tube body fixing structure in an endoscope cleaning sheath according to claim 1, wherein the extension portion is fixedly arranged in a communication groove provided on an outer side of the first fixing member or an inner side of the second fixing member.

7. The tube body fixing structure in an endoscope cleaning sheath according to claim 1, wherein the fitting portion includes a communication hole that brings the main lumen and an outer side of the first fixing member in communication with each other.

8. An endoscope cleaning sheath comprising the tube body fixing structure in an endoscope cleaning sheath according to claim 7,
wherein an insertion portion of an endoscope can be inserted in the main lumen via the communication hole, and a distal end nozzle portion that includes a spray nozzle that can feed a liquid to a front-side outer surface of the endoscope and is communicably connected to the sub-lumen is provided at the distal end portion of the first tube body.

9. An endoscope system including an endoscope and a light source apparatus, the endoscope system comprising:
the endoscope cleaning sheath according to claim 8; and
a tubular member through which the endoscope cleaning sheath is to be inserted.
